(19) United States
(12) Patent Application Publication
(10) Pub. No.: US 2016/0251698 A1
(43) Pub. Date: Sep. 1, 2016

(54) Analysis Unit for Carrying Out a Polymerase Chain Reaction, Analysis Device, Method for Operating such an Analysis Unit, and Method for Producing such an Analysis Unit

(71) Applicant: ROBERT BOSCH GMBH, Stuttgart (DE)

(72) Inventors: Franz Laermer, Weil Der Stadt (DE); Juergen Steigert, Stuttgart (DE); Jochen Hoffman, Leonberg (DE)

(21) Appl. No.: 15/030,562

(22) PCT Filed: Oct. 6, 2014
(86) PCT No.: PCT/EP2014/071320

§ 371 Date: Apr. 19, 2016

(30) Foreign Application Priority Data
Oct. 23, 2013 (DE) 10 2013 221 525.8

(51) Int. Cl. C12Q 1/68; B01L 7/00; G01N 35/00; B01L 3/00

(52) U.S. Cl. CPC C12Q 1/686; B01L 3/502715; B01L 7/52; G01N 35/00871; B01L 2300/044; B01L 2300/046; B01L 2300/0627; B01L 2300/18; B01L 2300/0861; B01L 2300/08; G01N 2035/00237; B01L 2300/14

(57) ABSTRACT

An analysis unit is configured to carry out a polymerase chain reaction. The analysis unit includes a lid element with at least one lid recess and a base element with at least one base recess. The base recess is arranged opposite the lid recess to form a reaction chamber. The analysis unit further includes a film arranged, at least in the region of the lid recess, between the lid element and the base element. The analysis unit also includes at least one channel formed between the lid element and the base element and configured to channel a fluid into and/or out of the base recess of the reaction chamber. The analysis unit includes a probe carrier arranged in the base recess and including at least one indicator material as the probe, for identifying a biochemical material. The indicator material on the probe carrier is in a solid aggregate state.

### PRIOR ART

The present invention relates to an analysis unit for carrying out a polymerase chain reaction, an analysis unit, a method for operating an analysis unit, a method for producing an analysis unit, and a corresponding computer software product.

Microfluidic diagnostic systems such as labs on a chip (LOCs) allow miniaturized and integrated execution of complex work flows for the specific detection of a wide variety of molecules. In publicly known LOC systems, the sample material to be analyzed is first amplified by means of PCR and then analyzed on a microarray (as described for example in J. Choi et al.: An integrated allele-specific polymerase chain reaction-microarray chip for multiplex single nucleotide polymorphism typing, Lab on a Chip, Vol. 12, 2012). The combination of these two operations and the multistep process control require prolonged processing time and complex process control.

### DISCLOSURE OF THE INVENTION

Against this backdrop, the present invention provides an analysis unit for carrying out a polymerase chain reaction, an analysis unit, a method for operating an analysis unit, methods for producing an analysis unit, and a corresponding computer software product according to the main claims.

Advantageous embodiments are presented in the respective dependent claims and the following description.

The present invention provides an analysis unit for carrying out a polymerase chain reaction, wherein the analysis device comprises the following features:
a lid element having at least one lid recess;
a base element having at least one base recess, wherein the base recess is arranged opposite the lid recess in order to form a reaction chamber;
a film which is arranged, at least in the area of the lid recess, between the lid element and the base element;
at least one channel which is formed between the lid element and the base element in order to channel a fluid into and/or out of the base recess of the reaction chamber; and
a probe carrier arranged in the base recess which has at least one indicator material as a probe for identifying a biochemical material, wherein the indicator material on the probe carrier is in a solid aggregate state.

A lid element and/or a base element can be understood to refer e.g. to a component produced from a plastic, particularly a polymer. A recess can be understood to refer to a depression in the lid element or the base element. A film can be understood to refer to a flat flexible element such as a plastic layer. In this case, for example, the film may be fluid-impermeable. A probe carrier can be understood to refer to a stiff, particularly an element on which the indicator material is arranged and fixed. An indicator material can be understood to refer e.g. to a chemical reagent or a biomolecule which, on contact with a predetermined biochemical material, undergoes a change in state which can be identified and/or evaluated (e.g. by optical analysis). Identification can be understood e.g. as meaning recognition of the presence of a predetermined material per se and/or recognition of a quantity and/or a quality of the predetermined material.

The present invention is based on the finding that a solid-phase reaction in the area of biochemical analysis processes can be directly carried out in an individual reaction chamber in which a liquid phase reaction of a biochemical reaction process also takes place. As the indicator material is usually present as a solid phase, i.e. in a solid aggregate state, the entire biochemical reaction process can take place in a small and compact analysis unit. The starting material to be analyzed and any required catalytic materials which are required, e.g. for obtaining or synthesizing intermediate products of the biochemical reaction process, can in this case be channeled in the form of a fluid via the channel into the reaction chamber or the base recess.

The present invention is advantageous in that it opens up an extremely simple technical possibility for carrying out a biochemical reaction without having to manually bring together intermediate products of the biochemical reaction from different process stages. In particular, it obviates the need to manually place an intermediate product of a first partial reaction, in which a liquid-phase reaction is carried out, in a separate compartment in order to carry out a second partial reaction in which a solid-phase reaction takes place. The present invention therefore allows a desired biochemical reaction to be carried out much less expensively and more quickly, particularly if this biochemical reaction consists of two separate parts or partial reactions based on materials in different aggregate states.

An embodiment of the present invention in which the film is attached to the lid element, and particularly in which the film seals an opening in the lid recess opposite the through opening in a fluid-impermeable manner and/or in which the lid element has a through opening in the area of the lid recess, is advantageous. Such an embodiment of the present invention is advantageous in that a fluid or another material is very easily (further) movable into the base recess or the reaction chamber, for example in order to channel it out of the base recess or the reaction chamber and/or force it into a further reaction chamber. For example, this movement of the fluid or the other materials is inducible by pressure on the lid element in the area of the lid recess which is transferred via the film to the fluid in the base recess. Such movement of the fluid or the other materials is quite easily inducible if the lid element has a through opening in the area of the lid recess via which pneumatic pressure is transferred to the lid recess, thus exerting pressure on the film, which as a result bulges outward in the area of the base recess.

According to a particularly favorable embodiment of the present invention, the lid element may comprise at least one further lid recess, and the base element may comprise at least one further base recess, wherein the further lid recess is arranged opposite the further base recess, and wherein the base recess and the further base recess are fluidly interconnected by the channel. Such an embodiment of the present invention is advantageous in that two different reaction chambers can be provided, so that e.g. various process stages of the biochemical reactions, which for example require different reaction temperatures, can be carried out in different areas of the analysis unit (i.e. different reaction chambers). This advantageously allows very flexible use of the analysis unit for various biochemical reactions.

According to a further embodiment of the present invention, in order to allow particularly flexible and precise detection of individual biochemical materials which for example require a reaction with an indicator material at various reaction temperatures, a further probe carrier can be arranged in the further base recess which has at least one further indicator material as a further probe for identification of another biochemical material, wherein the further indicator material on the further probe carrier is in a solid aggregate state.

According to a further embodiment of the present invention, the base recess may also divide the channel into at least two partial channels, wherein at least one partial channel is fluidly connectable to the external environment of the analysis device, and particularly wherein at least one partial channel has a valve. Such an embodiment of the present invention provides the advantage of particularly simple and/or controllable filling or removal of material, particularly a fluid material, from or into the base recess or the reaction chamber.

In a particularly advantageous embodiment of the present invention, in order to allow cyclic repetition of a plurality of process stages in particular, the partial channels are fluidly interconnected or interconnectable at an end facing away from the base recess, and in this embodiment in particular, the partial channels are components of a ring-shaped channel. Such an embodiment of the present invention is advantageous in that a biochemical reaction can take place, for example, in various base recesses or reaction chambers by means of progressive cyclical displacement via the partial channels, obviating the need to change the direction of the reaction.

An embodiment of the present invention in which the probe carrier comprises at least one further indicator material as a probe for identifying at least one further biochemical material, wherein the further indicator material on the probe carrier is in a solid aggregate state, is particularly advantageous. Such an embodiment of the present invention is advantageous in that a plurality of different biochemical materials and/or different concentrations of such materials are identifiable or detectable in a particularly economical, rapid, and easily technically realizable manner on a single probe carrier.

According to an advantageous embodiment of the present invention, in order to ensure that an indicator material on the probe carrier remains unchanged in its arrangement at a predetermined position, the probe carrier may have at least one probe carrier recess in which the at least one indicator material is arranged, and particularly in which the probe carrier recess has a predetermined minimum depth.

Moreover, according to an embodiment, the probe carrier may also be configured and arranged in such a manner that during the polymerase chain reaction, a reaction of a substance with the indicator material for identifying the biochemical material is feasible. Such an embodiment of the present invention provides the advantage of a particularly compact design of the analysis unit, wherein the analysis unit is also producible in a highly economical manner.

The present invention also provides an analysis device for operating an analysis unit according to a variant presented herein, said analysis device comprising at least the following features:
a holder for the placement and/or holding of the analysis unit during operation of the analysis device;
a temperature control unit for controlling the temperature of a fluid or a solid in the analysis unit held by the holder; and
an evaluation unit for evaluating a change in the indicator material.

A holder can be understood to refer e.g. to a supporting surface on which the analysis unit is placed during operation of the analysis device. A temperature control unit can be understood to refer to a unit which heats or cools the at least one partial section of the analysis unit. An evaluation unit can be understood to refer to a unit which detects a change in state of the at least one indicator material, for example by optical analysis of electromagnetic radiation converted or emitted by the indicator material (or an indicator modified by the biochemical material). Such an embodiment of the present invention is advantageous in that it allows highly compact analysis of a biochemical material with a simple and easily-produced analysis unit.

An embodiment of the present invention in which the temperature control unit is formed so as to simultaneously adjust each of the different sections of the analysis unit to a different temperature is particularly advantageous. Such an embodiment of the present invention is advantageous in that different partial reactions, which require different reaction temperatures, can be carried out simultaneously in different sections of the analysis unit. This makes it possible to very quickly carry out analysis of a biochemical material with an analysis unit that is technically simple to produce.

Moreover, the invention provides a method for operating an analysis unit according to a variant presented herein, said method comprising the following steps:
provision of the analysis unit;
application to the analysis unit of a material to be analyzed and/or a catalytic material for carrying out a reaction in the analysis unit; and evaluation of a change in the indicator material on the probe carrier.

Such an embodiment of the present invention is advantageous in that it allows analysis of a biotechnical material to be carried out in a particularly rapid and economical manner.

An embodiment of the present invention in which the steps of application and evaluation can be at least partially carried out simultaneously is also advantageous. Such an embodiment of the present invention is advantageous, for example, in that various partial steps of the polymerase chain reaction can be carried out simultaneously or in parallel, so that a result of the polymerase chain reaction can be analyzed very quickly on the one hand, and the polymerase chain reaction can be carried out in a very simple technical manner, i.e. particularly without intermediate manual steps, on the other.

According to a further embodiment of the present invention, in the step of application and/or evaluation, the material to be analyzed, the catalytic material, and/or the indicator material can be temperature-controlled. Such an embodiment of the present invention is advantageous in that different reaction temperatures can be used in the various partial steps of the polymerase chain reaction, allowing the individual partial steps of this polymerase chain reaction to take place in an environmental scenario that is optimal for them.

Moreover, the present invention also provides a method for producing an analysis unit according to a variant presented herein, said method comprising the following steps:
provision of the lid element having at least one lid recess, wherein the lid element has a through opening in the area of the lid recess, the base element having at least one base recess, the film, and a probe carrier which has at least one indicator material as a probe for identifying a biochemical material, wherein the indicator material on the probe carrier is in a solid aggregate state;
arrangement of the probe carrier in the base recess;
covering of the lid recess with the film; and
formation of the channel area in order to channel fluid into and/or out of the base recess of the reaction chamber, wherein said formation is carried out by arranging the base recess opposite the lid recess in order to form the reaction chamber.

Such an embodiment of the present invention is advantageous in that it enables the production of an analysis unit for particularly economical and rapid identification of a biochemical material.

The present invention further provides a controller which is configured to carry out or implement the steps of a variant of a method presented herein in corresponding devices. The object of the invention is also rapidly and efficiently achievable by means of these variant embodiments of the invention in the form of a controller.

In the present invention, a controller can be understood to refer to an electrical device which processes sensor signals and emits control and/or data signals based thereon. The controller may have an interface which may be configured in hardware and/or software form. In a hardware configuration, for example, the interface may be part of a so-called system ASIC, which comprises a wide variety of functions of the controller. However, it is also possible for the interfaces themselves to constitute integrated circuits or to be at least partially composed of discrete components. In a software configuration, the interfaces may be software modules, which for example are present on a microcontroller in addition to other software modules.

A computer software product with program code which can be stored on a machine-readable medium such as a semiconductor storage unit, a hard drive storage unit, or an optical storage unit, and which is used for carrying out the method according to one of the embodiments described above when the software product is run on a computer or a device, is also advantageous. In particular, the present invention provides a computer software product with program code for conducting or controlling the steps of a method presented herein when the software product is run on a device.

The present invention further provides a device which is configured to carry out or implement the steps of a variant of a method presented herein in corresponding devices. The object of the invention is also rapidly and efficiently achievable by means of these variant embodiments of the invention in the form of a device.

In the present invention, a device can be understood to refer to an electrical device which processes sensor signals and emits control and/or data signals based thereon. The device may have an interface which can be configured as hardware and/or software. In a hardware configuration, the interfaces may be part of a so-called system ASIC, which comprises a wide variety of functions of the device. However, it is also possible for the interfaces themselves to constitute integrated circuits or to be at least partially composed of discrete components. In a software configuration, the interfaces may be software modules, which for example are present on a microcontroller in addition to other software modules.

In the following, by way of example, the invention is explained in greater detail with reference to the attached drawings. The figures show the following:

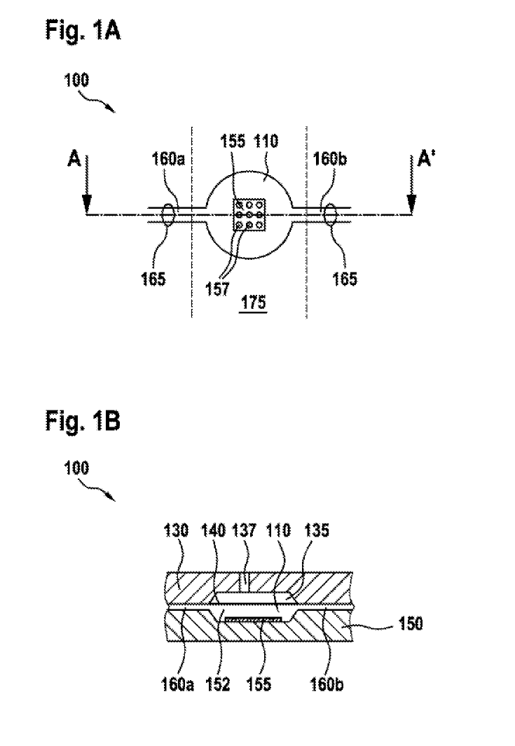

FIG. 1A is a top view of an analysis unit according to an embodiment of the present invention;

FIG. 1 B is a sectional view of an analysis unit according to the embodiment of the invention shown in FIG. 1A;

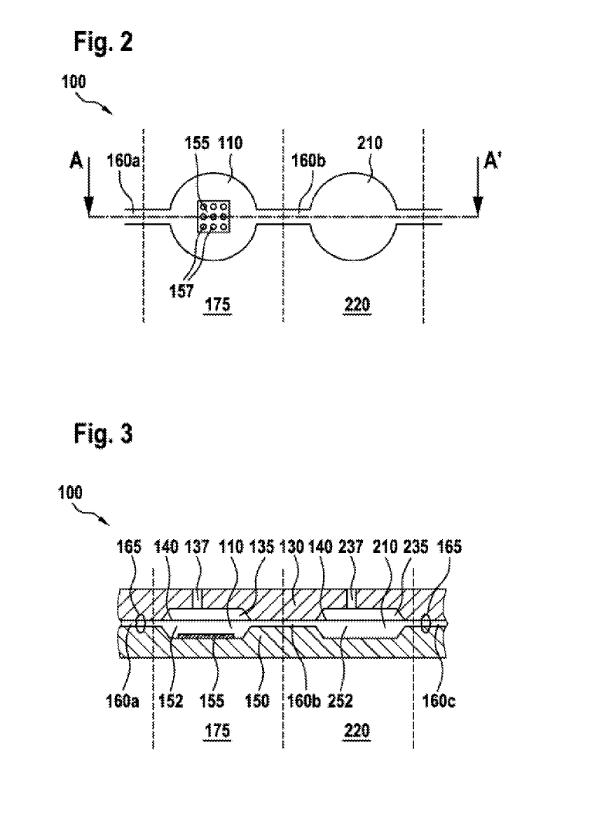

FIG. 2 is a top view of an analysis unit according to a further embodiment of the present invention;

FIG. 3 is a sectional view of an analysis unit according to the embodiment of the present invention shown in FIG. 2;

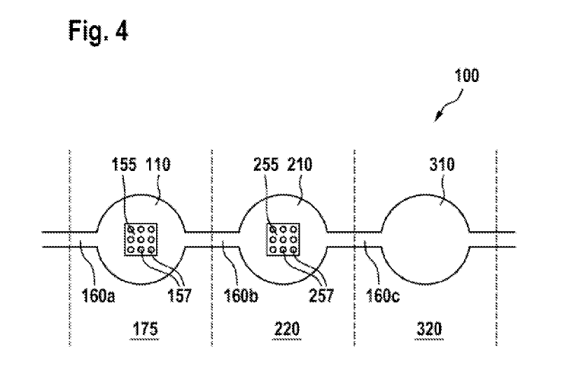

FIG. 4 is a top view of an analysis unit according to a further embodiment of the present invention;

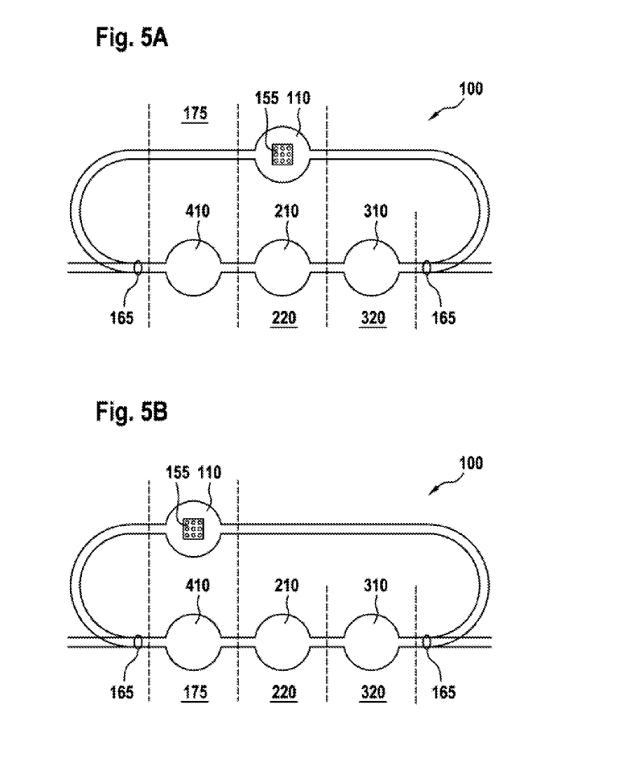

FIGS. 5A and 5B are top views of analysis units according to further embodiments of the present invention;

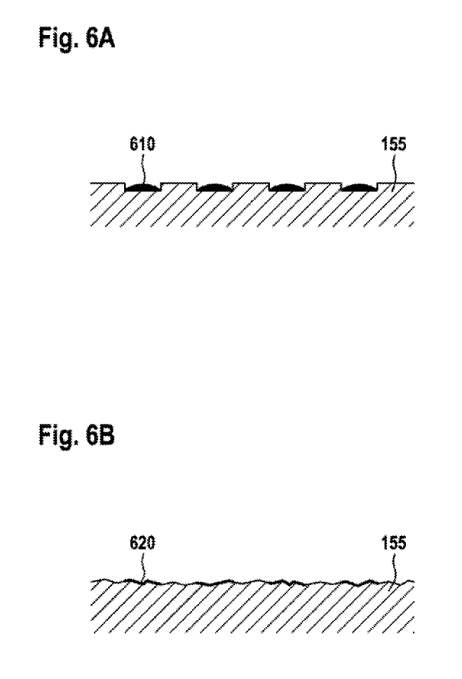

FIG. 6A is a sectional view of a probe carrier according to an embodiment of the present invention;

FIG. 6B is a sectional view of a probe carrier according to a further embodiment of the present invention;

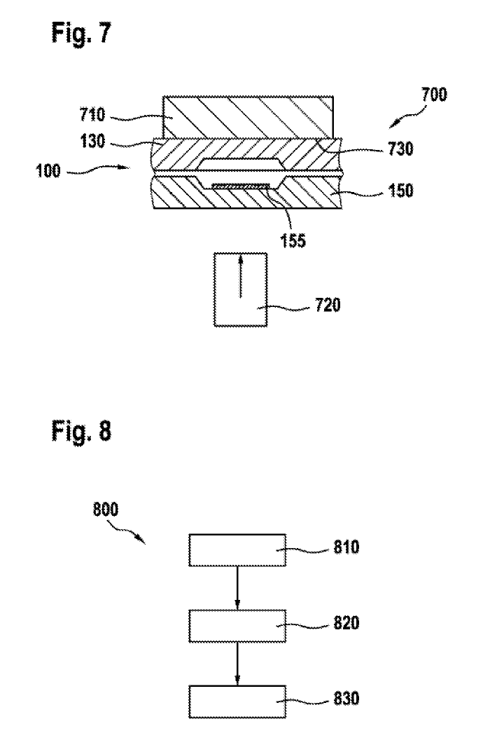

FIG. 7 is a schematic view of an analysis device including a schematic view of an analysis unit in the analysis device;

FIG. 8 is a flow chart of an embodiment of a method for operating an analysis unit; and

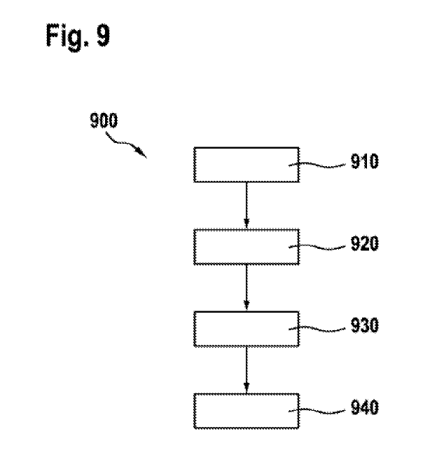

FIG. 9 is a flow chart of an embodiment of a method for producing an analysis unit.

In the following description of advantageous embodiments of the present invention, the same or similar reference numbers are used for the elements shown in the various figures which have similar functions, with a repeated description of said elements being dispensed with.

In the biochemical method presented herein, the DNA microarray (as an example of a probe carrier) is directly integrated into a PCR reaction chamber. The DNA probes immobilized on the DNA microarray play an active role in the PCR, thus immobilizing specific amplification products (i.e. the biochemical material).

The surface-bound products (or intermediate products) from a biochemical process (such as PCR) may be detected and/or evaluated either in real time or after the reaction.

FIG. 1A shows a top view of an analysis unit 100 according to an embodiment of the present invention. FIG. 1B shows a sectional view of the embodiment of the present invention shown in FIG. 1A along line AA′ in FIG. 1A. In this case, the analysis unit 100 has only a single PCR reaction 110.

The analysis unit 100 comprises a plurality of layers, as can be seen from FIG. 1B. A layer structure is composed of a first polymer substrate as a lid element 130 which comprises a lid recess 135 and a through hole 137, wherein a deformable polymer membrane is formed as a film 140 on the lower side of the lid element 130.

This film 140 may, for example, be connected to the lid element 130 in a fluid-impermeable manner so that contact with a medium which is movable through the through hole does not take place in an area outside the film 140.

A base element 150, for example in the form of a second polymer substrate which has a DNA microarray as a probe carrier 155 in a base element recess 152 of the PCR reaction chamber 110, is configured plane-parallel to this structure of the lid element 130. Various oligonucleotides are immobilized on the microarray 155 in the form of individual spots as probes 157. For filling and emptying, one microfluidic channel 160 each (divided for example by the reaction chamber 110 into two partial channels 160a and 160b) is connected to the chamber 110. The two channels 160a and 160b have one controllable valve 165 each in order to keep the chamber 110, which is filled with a PCR reaction mix (i.e. material to be analyzed and/or catalytic material required as an auxiliary for carrying out the biochemical reaction), sealed during the (biochemical) reaction. In a specified area 175 in which the reaction chamber 110 is arranged, thermal energy for carrying out PCR can be applied and removed by means of an analysis device described in further detail below.

During PCR, certain DNA motifs of a template DNA are first amplified in the liquid phase. The PCR products formed can now bond to section-specific immobilized oligonucleotides. Identification of the spots to which a PCR product has bonded allows the presence/absence of specified DNA to be determined.

FIG. 2 shows a top view of an analysis unit 100 according to an embodiment of the present invention. FIG. 3 shows a sectional view along section line AA′ of the embodiment of the analysis unit 100 shown in FIG. 2, which has two PCR reaction chambers 110 and 210 fluidly interconnected via the partial channel 160b. In this case, the second reaction chamber 210 can be formed analogously to the (first) reaction chamber (except for the presence of the probe carrier 155). For example, in order to form the second reaction chamber 210, the lid element 130 may have a further lid recess 235 which is fluidly connected via a further through hole 237 to an outer environment of the analysis device 100. The film may also seal the second lid recess 235 in a fluid-impermeable manner on a side opposite the further through hole. The base element 150 may have a further base recess 252 by means of which the second reaction chamber 210 is formed.

In this embodiment, there are two specified temperature ranges 175 and 220 in each of which one constant but different temperature is set for the entire duration of the PCR. This allows the requirement for a temperature control unit of an analysis device, as compared to an analysis device for operating the analysis unit 100 according to the embodiment shown in FIGS. 1 A and 1 B, to be reduced, with the requirement being to vary the temperature between −60° C. and −95° C. within a time window of minutes (e.g. by means of a Peltier element). In the embodiment shown in FIGS. 2 and 3, in order to expose the PCR reaction mixture to various temperatures, the reaction volume (i.e. the fluid containing the material to be analyzed and/or the catalytic material) is moved back and forth between the two chambers by means of the deformable polymer membrane 140. This back and forth movement may be produced, for example, by means of pressure applied via the through holes 137 or 237 to the film 140 by the respective reaction chambers 110 or 210, with the outward bulging of the film in these reaction chambers 110 or 210 causing the fluids located in the respective reaction chambers to be channeled out.

An important objective of the present invention can be considered to be opening up the possibility of suitable structures and processes for carrying out PCR in a polymeric multilayer composite. In this manner, the processing time is decreased, and the number of steps, for example in detection of nucleic acids, is reduced.

In this case, a polymeric layer structure of the analysis unit 100 with an integrated DNA microarray 155 may be used. The DNA microarray 155 is either directly present in a PCR reaction chamber 110 or present in a separate array chamber. A PCR reaction is carried out by moving the reaction volume (i.e. the fluid containing the material to be analyzed or the catalytic material) back and forth between chambers 110 or 210, which are at different temperatures. As probes 157 of the microarray 155, the immobilized oligonucleotides play an active role in the PCR reaction so that DNA sequences are localized at specified positions of the array 155.

The present invention provides the following advantages, with PCR as an example of a biochemical reaction:
The PCR product no longer has to be rebuffered before hybridization.
The LoC system no longer requires buffer for hybridization.
The structures and methods described allow a faster process together with simplified process control.
The total duration of detection of genetic features is reduced. In the present method, PCR products can even be immobilized at specific spots of the microarray during PCR in a site-resolved and detectable manner.
A surface-sensitive measuring method is used.

According to a further embodiment of an analysis unit 100, the reaction takes place in a system having three PCR chambers 110, 210, and 310 which are fluidly interconnected via partial channels 160, as shown in the top view of FIG. 4. In this case, the three analysis chambers 110, 210, and 310 are similarly configured, with one probe carrier 255 being arranged in each of the first two analysis chambers 110 and 210. For three-step PCR, this allows three different temperature-controlled areas/chambers to be configured, for example in three-step PCR reactions (e.g. annealing temperature 175=50° C.-65° C., extension temperature 220=72° C., denaturation temperature 320=95° C.). In this case, the array 155 or 255 may be positioned either in the chamber 110 at annealing temperature 175 or in the chamber 210 at extension temperature 220. Moreover, the first reaction chamber 110 and the second reaction chamber 310 may also be controlled to a temperature of between 55 and 72° C. (annealing and extension temperature), while the chamber 210 between them may be controlled to denaturation temperature.

For two-step PCR, the reaction volumes may first (using partial channel 160b) be moved back and forth between chambers 110 and 210 at 1 to 40 cycles, after which they are moved back and forth (using partial channel 160c) between chambers 210 and 310 at 1 to 40 cycles, for example with only chamber 310 containing the (second) array 225, in contrast to what is shown in FIG. 4.

FIG. 5A shows a top view of an analysis unit 110 according to a further embodiment of the present invention, in which a reaction chamber 110 (array chamber) contains the array 155. The chamber 110 is either in the temperature range of the annealing temperature 175 or the extension temperature 220 (as can be seen, for example, in the top view of FIG. 5B). This embodiment makes it possible to first carry out PCR in the PCR chambers 410, 210 or 310 without the array 155. After amplification, the reaction mix is then transferred to the array chamber 110 so that the PCR products can be specifically bonded to oligonucleotides 157. This bonding may take place by hybridization or the primer extension reaction, for example by moving the reaction volume back and forth between a chamber at denaturation temperature and the array chamber 110.

According to a further embodiment, the chamber inlet 160a and outlet 160b may have a fluid layout provided with non-return valves 165 as shown in FIGS. 2, 4, 5A, and 5B. In this case, a (single) pump direction is preset for volume displacement, which simplifies actuation of the fluid.

FIGS. 6A and 6B each show a structure for protecting the surface of the array 155 (particularly the spots of the immobilized oligonucleotides as probes 157). As the polymer membrane 140 is pneumatically deformed and thus pressed in the direction of the bottom of the base recess 152 in order to transfer the PCR reaction volume from one PCR chamber 110 to another (according to FIG. 2 and FIG. 4), the oligonucleotides 157 immobilized in the array format 155 may possibly be damaged. As a countermeasure, the spots of the array 155 may be immobilized either in depressions 610 or on a microscopically rough surface 620.

The oligonucleotides 157 may be fixed on a wide variety of materials on the probe carrier 155 using any of the immobilization chemistries known from prior art, such as 3-aminopropyltriethoxysilane (APTES), 1-ethyl-3(3-dimethylaminopropyl)-carbodiimide (EDC), 1,4-phenylene diisothiocyanate (PDITC), s-SIAB, s-MBS, s-GMBS or s-MBP. The arrays 155 may be immobilized either directly on a polymer substrate (i.e. the base element 130) or spotted onto a substrate 155, which is then placed in a PCR chamber 110 and fixed in a second step.

FIG. 7 shows a section through an embodiment of an analysis device 700 for operating an analysis unit 100 according to an embodiment of the present invention having a polymer layer structure. The analysis device 700 has a heating or temperature control element 710 (temperature control unit) configured on its upper side and a detection unit 720 (evaluation unit) configured on its lower side. The arrangement of the temperature control element 710 and the detection unit 720 may also be reversed. The temperature control element 710 may also comprise a plurality of partial units in which the adjacently placed areas of an analysis unit 100 are simultaneously adjusted to different temperatures. Thermal energy for carrying out temperature-controlled PCR can be provided by the temperature control unit 710 by methods such as using temperature-controlled heating elements, Peltier elements, infrared heaters or convection. In order to hold the analysis unit 100, a holder 730 may also be provided which fixes the analysis unit 100 in place, e.g. on a surface of the temperature control unit 710, during operation of the analysis device.

The PCR products bound to a microarray 155 can be identified in the detection unit 720 either during or after the reaction by various methods:

1. Fluorometric detection. During the PCR, fluorophores are incorporated into the PCR products produced. After excitation of these fluorophores (e.g. by evanescent fields, confocally, or by means of transmitted light), the fluorescence emitted can be measured using a CCD camera, CMOS chips, or a photomultiplier.

2. Detection of surface plasmons. The oligonucleotides 157 are immobilized, for example on gold spots. Surface plasmons in the spots are excited after or during PCR. The plasmon resonance frequency shifts based on the number of DNA molecules near the surface. Findings on binding events can be obtained by measuring and evaluating the intensities and/or frequencies emitted by the spots.

The reaction mix contains the components polymerase, reaction buffer, PCR primers and nucleotides, as well as reaction-improving components such as BSA and/or Tween. If biotin-dUTP nucleotides are used in PCR, the PCR products produced are labelled with biotin molecules, to which fluorophore-streptavidin conjugates can bond in a further step. Alternatively, fluorophore-labeled primers may be used in PCR so that the PCR products generated are directly labeled with a fluorophore. The two PCR primers may be used in different concentrations to carry out asymmetric PCR. The required structures in the polymer substrates may be produced e.g. by milling, injection moulding, hot stamping, or laser structuring. The microarray 155 may either be formed directly in the polymer or integrated into the polymer layer structure as an insert component, e.g. produced from glass.

Material Examples:

Polymer substrate (e.g. for the lid 130 and base element 150): thermoplastics (e.g., PC, PP, PE, PMMA, COP, COC, PEEK)

Polymer membrane (film 140): elastomer, thermoplastic elastomer TPU, TPS, thermoplastics, hot-sealing films, sealing films for microtiter plates, latex

Example Conformation of the Biomolecules:
Length of oligonucleotides 157: 5-100 nucleotides
Linker molecules: thiol groups, amino groups, gold, glutaraldehyde, acrydite, e.g. linked to the oligonucleotide by a carbon chain
PCR primer: length between 5 and 100 nucleotides, melting temperatures of the two primers may differ widely
Polymerase: hot-start polymerases, proofreading polymerases
Spot diameter: 1-500 μm

Example Dimensions of the Embodiments:
Thick polymer substrate: 0.5 to 5 mm
Channel diameter in polymer substrates: 10 μm to 3 mm
Thickness of polymer membrane: 5 to 500 μm
Volume of cavities in the polymer substrates: 1 mm³ to 1000 mm³

Example pressures for the actuation of a polymer membrane:
0.2 bar-2 bar

The invention may be used for analytic systems, particularly for microfluidic lab on chip systems for environmental analysis or medical diagnosis.

FIG. 8 shows a flow chart of an embodiment of a method 800 for operating an analysis unit. The method 800 comprises a step 810 of providing the analysis unit and a step 820 of application to the analysis unit of a material to be analyzed and/or a catalytic material for carrying out a reaction in the analysis unit. Finally, the method 800 comprises a step 830 of evaluation of a change in the indicator material on the probe carrier.

FIG. 9 shows a flow chart of an embodiment of a method 900 for producing an analysis unit. The method 900 includes a step 910 of providing the lid element comprising at least one lid recess, wherein the lid element has a through opening in the area of the lid recess, the base element having at least one base recess, the film, and a probe carrier, which has at least one indicator material as a probe for identifying a biochemical material, wherein the indicator material on the probe carrier is in a solid aggregate state. Moreover, the method 900 also comprises a step 920 of arranging the probe carrier in the base recess and a step 930 of covering the lid recess with the film. Finally, the method 900 comprises a step 940 of forming the channel area in order to channel a fluid into and/or out of the base recess of the reaction chamber, wherein said formation takes place by arrangement of the base recess opposite the lid recess in order to form the reaction chamber.

The embodiments described and shown in the figures are selected solely by way of example. Different embodiments may be combined with one another as a whole or with respect to individual features. An embodiment may also be supplemented by features of another embodiment.

Moreover, the process steps according to the invention may be repeated or carried out in an order other than described above.

If an embodiment includes the connecting phrase “and/or” between a first feature and a second feature, this is to be interpreted as indicating that the embodiment according to one variant comprises both the first feature and the second feature, and the embodiment according to another variant comprises either the first feature alone or the second feature alone.

1. An analysis unit configured to carry out a polymerase chain reaction, the analysis unit comprising:
a lid element having at least one lid recess;
a base element having at least one base recess, the at least one base recess is arranged opposite the at least one lid recess to form a reaction chamber;
a film arranged, at least in an area of the at least one lid recess, between the lid element and the base element;
at least one channel formed between the lid element and the base element and configured to channel a fluid into and/or out of the at least one base recess of the reaction chamber; and
a probe carrier arranged in the at least one base recess and including at least one indicator material as a probe configured to identify a biochemical material,
wherein the at least one indicator material on the probe carrier is in a solid aggregate state.

2. The analysis unit according to claim 1, wherein:
the lid element includes a through opening in the area of the at least one lid recess; and
the film is attached to the lid element to seal an opening in the at least one lid recess opposite the through opening in a fluid-impermeable manner.

3. The analysis unit according to claim 1, wherein:
the lid element has at least one further lid recess;
the base element has at least one further base recess;
the at least one further lid recess is arranged opposite the at least one further base recess; and
the at least one base recess and the at least one further base recess are fluidly connected by the at least one channel.

4. The analysis unit according to claim 3, further comprising:
a further probe carrier is arranged in the at least one further base recess, the further probe carrier including, as a further probe, at least one further indicator material configured to identify another biochemical material,
wherein the at least one further indicator material on the further probe carrier is in in a solid aggregate state.

5. The analysis unit according to claim 1, wherein:
the at least one base recess divides the at least one channel into at least two partial channels; and
at least one of the at least two partial channels is configured to allow a fluid connection with an external environment of the analysis unit.

6. The analysis unit according to claim 5, wherein the at least two partial channels are fluidly connected or connectable to one another at an end facing away from the at least one base recess.

7. The analysis unit according to claim 1, wherein the probe carrier is formed and arranged such a manner that during the polymerase chain reaction, a reaction of a substance with the at least one indicator material is carried out.

8. An analysis device configured to operate an analysis unit, which is configured to carry out a polymerase chain reaction, the analysis unit including a lid element having at least one lid recess, a base element having at least one base recess arranged opposite the at least one lid recess to form a reaction chamber, a film arranged between the lid element and the base element, at least one channel formed between the lid element and the base element and configured to channel a fluid into and/or out of the at least one base recess of the reaction chamber, and a probe carrier arranged in the at least one base recess and including at least one indicator material as a probe configured to identify a biochemical material, the analysis device comprising:
a holder configured to place and/or hold the analysis unit during operation of the analysis device;
a temperature control unit configured to control a temperature of a fluid or a solid in the holder held by the analysis unit; and
an evaluation unit configured to evaluate a change in the at least one indicator material.

9. The analysis device according to claim 8, wherein the temperature control unit is formed so as to simultaneously adjust each of a plurality of different sections of the analysis unit to a different temperature respectively.

10. A method for operating an analysis unit, which is configured to carry out a polymerase chain reaction, the analysis unit including a lid element having at least one lid recess, a base element having at least one base recess arranged opposite the at least one lid recess to form a reaction chamber, a film arranged between the lid element and the base element, at least one channel formed between the lid element and the base element and configured to channel a fluid into and/or out of the at least one base recess of the reaction chamber, and a probe carrier arranged in the at least one base recess and including at least one indicator material as a probe configured to identify a biochemical material, the method comprising:
providing of the analysis unit;
applying to the analysis unit at least one of a material to be analyzed and a catalytic material for carrying out a reaction in the analysis unit; and
evaluating a change in the indicator material on the probe carrier.

11. The method according to claim 10, wherein applying the material and evaluating the change are at least partially carried out simultaneously.

12. The method according to claim 10, further comprising: controlling a temperature of, at least one of the material to be analyzed, the catalytic materials, and the indicator material during at least one of applying the material and evaluating the change.

13. A method for producing an analysis unit, which is configured to carry out a polymerase chain reaction, the analysis unit a base element having at least one base recess arranged opposite the at least one lid recess to form a reaction chamber, a film arranged between the lid element and the base element, at least one channel formed between the lid element and the base element and configured to channel a fluid into and/or out of the at least one base recess of the reaction chamber, and a probe carrier arranged in the at least one base recess and including at least one indicator material as a probe configured to identify a biochemical material, comprising:
providing:
a lid element having at least one lid recess, and a through opening in an area of the at least one lid recess;
a base element having at least one base recess;
a film; and
a probe carrier including at least one indicator material as a probe configured to identify a biochemical material, the indicator material on the probe carrier in a solid aggregate state;
arranging the probe carrier in the at least one base recess;
covering of the at least one lid recess with the film; and
forming a channel area between the lid element and the base element to channel a fluid into and/or out of the at least one base recess of a reaction chamber by arranging the at least one base recess opposite the at least one lid recess to form the reaction chamber.

14. The method of claim 10, further comprising controlling and/or implementing at least one of the providing, the applying, and the evaluating with a controller having units.

15. The method of claim 14, further comprising carrying out the controlling and/or the implementing with a computer software product with program code when the software product is run on a device.

16. The analysis unit according to claim 5, wherein:
the at least one of the at least two partial channels has a valve configured to allow the fluid connection with the external environment of the analysis unit.

17. The analysis unit according to claim 6, wherein:
the at least two partial channels are configured as components of a ring-shaped channel.
